(19) United States
(12) Patent Application Publication
(10) Pub. No.: US 2021/0038861 A1
(43) Pub. Date: Feb. 11, 2021

(54) SELF-CLEANING CATHETER SYSTEMS WITH SELF-MONITORING CAPABILITIES

(71) Applicant: MICROBOT MEDICAL LTD., Caesarea (IL)

(72) Inventors: Or SAMOOCHA, Bustan Hagalil (IL); Simon SHARON, Maayan Zvi (IL); Yosef PORAT, Afula (IL)

(21) Appl. No.: 16/965,976

(22) PCT Filed: Jan. 31, 2019
(86) PCT No.: PCT/IL2019/050123

§ 371 Date: Jul. 29, 2020

Related U.S. Application Data
(60) Provisional application No. 62/625,928, filed on Feb. 2, 2018.
(60) Provisional application No. 62/767,613, filed on Nov. 15, 2018.
(60) Provisional application No. 62/784,729, filed on Dec. 25, 2018.

(51) Int. Cl. A61M 25/00

(52) U.S. Cl. CPC A61M 25/0017; A61M 2025/0019; A61M 2205/04; A61M 2205/3306; A61M 2205/3507; A61M 2205/3317; A61M 2205/18

(57) ABSTRACT

Disclosed is s self-cleaning catheter system with self-monitoring capabilities. The catheter system includes a catheter configured to be implanted in a body cavity of a subject, a cleaning unit configured for motion within the catheter such as to mechanically prevent and/or remove or mitigate blockage of at least a section of the catheter, an implantable sensor, and an implantable controller functionally associated with the cleaning unit and configured for activation thereof. The implantable sensor is communicatively associated with the implantable controller and is configured to detect motion of the cleaning unit, and to output one or more signals indicative of the motion of the cleaning unit. The implantable controller is configured to receive the one or more signals from the implantable sensor and, based, at least, thereon, to provide at least one motion indication, at least when the one or more signals are indicative of malfunctioning of the cleaning unit.

### TECHNICAL FIELD

The present disclosure relates generally to self-cleaning catheter systems for fluid delivery, drainage, and/or passage.

### BACKGROUND

Shunts are often used as internal medical devices to drain aberrant fluids from different organs. FIG. 1A schematically depicts a prior art cerebral shunt 15 for draining cerebrospinal fluid (CSF) implanted in an infant patient 25. Shunt 15 includes a ventricular catheter 35, a drain tube 37, and a valve 39 regulating the flow of fluid from ventricular catheter 35 to drain tube 37. Ventricular catheter 35 is implanted in a brain ventricle (not indicated). FIG. 1B is a close-up view of ventricular catheter 35. A catheter head 41 of ventricular catheter 35 includes a plurality of apertures 47 and 49, along its length; the apertures often having different sizes and different spacings, such that CSF accumulated around ventricular catheter 35 drains through the apertures into drain tube 37, and away from the brain ventricle. The excess CSF is generally drained into a body cavity such as the abdomen. Ventricular catheter 35 may have length calibrations imprinted thereon, so that the surgeon can estimate how far ventricular catheter 35 has been inserted into the cranial cavity. Drain tube 37 is generally implanted just beneath the skin, with access to the cranial region to be drained, and into the abdominal cavity, being achieved by means of small incisions 55 in the meninges and the peritoneum respectively. To allow the patient to grow into adulthood without having to replace the shunt, an end section 61 of drain tube 37 may be bundled up in the abdominal cavity, so that it can unravel as the patient grows.

Such prior art simple shunts, as described above, generally have two major problems: (i) the inlet apertures might get clogged, and (ii) the ventricular catheter might become contaminated and thereby potentially cause an infection. When the ventricular catheter becomes clogged (e.g. due to clogging of the inlet apertures), an attempt to remove it from the body by surgery should be made. In cases where it is impossible to remove, another ventricular catheter may be placed in parallel to the malfunctioning one. When the ventricular catheter is contaminated, it must be removed from the body by surgery. Surgeries of this kind are often high-risk procedures.

The simple prior art shunts depicted in FIGS. 1A and 1B have a significant drawback in that after some period of time inside the human body, living tissue growth may result in blockage of the apertures by the tissue. This tissue is generally the main cause of shunt blockage. When trying to withdraw the shunt by surgery, the ingrown tissue may tear, causing intraventricular bleeding, which might be life threatening.

### SUMMARY

Aspects of the disclosure, according to some embodiments thereof, relate generally to self-cleaning catheter systems for fluid delivery, drainage, and/or passage configured for self-monitoring of the performance thereof. More specifically, but not exclusively, aspects of the disclosure, according to some embodiments thereof, relate to self-cleaning catheter systems for fluid delivery, drainage, and/or passage configured for self-monitoring of the performance of a cleaning unit, housed within a catheter of the catheter system, and configured for implementing the self-cleaning of at least a portion of the catheter.

In the present disclosure, according to some embodiments, the self-cleaning is mechanically effected using a cleaning unit configured for motion within the catheter. Advantageously, a sensor is utilized to monitor the motion of the cleaning unit and to provide feedback on the performance thereof. Thus, a malfunction of the cleaning unit and, in particular, of moving parts thereof, and/or unremovable blockage may be detected in real time.

Thus, according to an aspect of some embodiments, there is provided a self-cleaning catheter system with self-monitoring capabilities, the catheter system including a catheter configured to be implanted in a body cavity of a subject, a cleaning unit configured for motion within the catheter such as to mechanically prevent and/or remove or mitigate blockage of at least a section of the catheter, an implantable sensor, and an implantable controller functionally associated with the cleaning unit and configured for activation thereof, wherein the implantable sensor is communicatively associated with the implantable controller and is configured to detect motion of the cleaning unit, when the cleaning unit is activated, and to output one or more signals indicative of the motion of the cleaning unit, and wherein the implantable controller is configured to receive the one or more signals from the implantable sensor and, based, at least, thereon, to provide at least one motion indication, at least when the one or more signals are indicative of malfunctioning of the cleaning unit. According to some embodiments, the malfunctioning of the cleaning unit includes the cleaning unit not moving.

According to some embodiments, the malfunctioning of the cleaning unit includes the cleaning unit not moving according to programmed motion commands and/or the cleaning unit being out of position.

According to some embodiments, the body cavity includes a ventricle.

According to some embodiments, the sensor and the controller are communicatively associated by wire.

According to some embodiments, the catheter includes the sensor. The sensor is housed in the catheter.

According to some embodiments, the sensor is embedded in walls of the catheter, in proximity to the cleaning unit.

According to some embodiments, the catheter includes the controller. The controller is housed in the catheter.

According to some embodiments, the catheter system further includes an implantable power source (e.g. a battery) for powering one or more of the controller, the cleaning unit, and the sensor.

According to some embodiments, the controller and the power source are both housed within an implantable casing.

According to some embodiments, the cleaning unit is automatically activated on a periodic basis.

According to some embodiments, the catheter system further includes an implantable power receiver configured for wireless power transfer from an external activation unit.

The power receiver is further configured to power one or more of the controller, the cleaning unit, and the sensor.

According to some embodiments, the controller and the power receiver are both housed within an implantable casing.

According to some embodiments, the power receiver is further configured to transmit the at least one motion indication to the external activation unit. The external activation unit is configured to generate an alert when the at least one motion indication indicates or specifies the malfunctioning of the cleaning unit.

According to some embodiments, the power receiver is further configured to transmit the at least one motion indication to an external system. The external system is configured to generate an alert when the at least one motion indication indicates or specifies the malfunctioning of the cleaning unit.

According to some embodiments, the controller includes a transmitter configured to transmit the at least one motion indication to the external activation unit. The external activation unit being configured to generate an alert when the at least one motion indication indicates or specifies the malfunctioning of the cleaning unit.

According to some embodiments, the controller includes a transmitter configured to transmit the at least one motion indication to an external system. The external system is configured to generate an alert when the at least one motion indication indicates or specifies the malfunctioning of the cleaning unit.

According to some embodiments, the alert further includes a notification that medical attention is required.

According to some embodiments, the controller includes processing circuitry configured to assess whether the cleaning unit is malfunctioning based, at least in part, on the one or more signals received from the sensor. The at least one motion indication specifies the assessment.

According to some embodiments, the controller includes processing circuitry configured to assess whether the cleaning unit is malfunctioning based, at least in part, on the one or more signals received from the sensor. The at least one motion indication is provided only when the assessment is of malfunctioning and specifies the assessment.

According to some embodiments, the one or more signals output by the sensor include at least two signals including a first signal and a later last signal. The processing circuitry is further configured to assess whether the cleaning unit is malfunctioning upon receiving the first signal, and, if the assessment based, at least in part, on the first signal, is of malfunctioning, to initiate corrective action. The processing circuitry is further configured to assess again whether the cleaning unit is malfunctioning upon receiving the last signal. The at least one motion indication specifies the (last) assessment based, at least in part, on the last signal.

According to some embodiments, the external system includes processing circuitry configured to assess whether the cleaning unit is malfunctioning based, at least in part, on the at least one motion indication.

According to some embodiments, the one or more signals output by the sensor include at least two signals including a first signal and a later signal. The at least one motion indication provided by the controller includes a first motion indication and a later last motion indication corresponding to the first signal and the later signal, respectively. The processing circuitry is further configured to assess whether the cleaning unit is malfunctioning upon receiving the first motion indication, and, if the assessment based, at least in part, on the first motion indication, is of malfunctioning, to initiate corrective action. The processing circuitry is further configured to assess again whether the cleaning unit is malfunctioning upon receiving the last motion indication, and to generate an alert when the (last) assessment based, at least in part, on the last motion indication, is of malfunctioning.

According to some embodiments, the processing circuitry is configured to compute, based on the at least one of the received one or more signals, an amplitude of oscillations of the cleaning unit and/or a mean position of the cleaning unit. The assessment(s) is based, at least in part, on the amplitude and/or mean position.

According to some embodiments, the corrective action includes one or more of increasing power supplied to the cleaning unit, changing a duty cycle of the cleaning unit, changing an activation waveform of the cleaning unit, changing a sampling rate of the sensor, and changing a sensitivity of the sensor.

According to some embodiments, wherein the sensor and the controller are communicatively associated by wire, wherein the power receiver is further configured for transmission as described above, or wherein the controller includes a transmitter as described above, the at least one motion indication includes or substantially includes the one or more motion signals in the form of at least one wireless signal.

According to some embodiments, the external system is one or more of a smartphone, smartwatch, tablet, laptop, or personal computer (PC).

According to some embodiments, the external system is a cloud computer.

According to some embodiments, the external system is a hospital/clinic computer.

According to some embodiments, the sensor is automatically activated each time the cleaning unit is activated.

According to some embodiments, the external system is, or includes, an external activation unit as described above.

According to some embodiments, the external system is a headset configured to be worn by the subject. The headset includes an external activation unit as described above, and at least one user interface configured to generate the alert and to allow the subject to activate the cleaning unit. The controller includes a power receiver as described above.

According to some embodiments, the sensor is configured to be manually activated.

According to some embodiments, the sensor is configured to be automatically activated upon activation of the cleaning unit.

According to some embodiments, the catheter includes a catheter tube and a catheter tip member fluidly connected to the catheter tube and housing the cleaning unit.

According to some embodiments, the catheter tip member includes one or more apertures fluidly coupling the catheter tube to the outside thereof. The cleaning unit is configured to at least one of prevent and mitigate blockage of the one or more apertures.

According to some embodiments, the cleaning unit includes an elongated shaft including one or more arms configured to project into the one or more apertures and to move therein.

According to some embodiments, the movement of the one or more arms is configured to prevent at least tissue from entering at least some of the one or more apertures when the catheter tip member is implanted within the body cavity.

According to some embodiments, the cleaning unit is configured to allow vibration thereof. The movement of the one or more arms within the one or more apertures may be induced by the vibration of the cleaning unit.

According to some embodiments, the one or more apertures include at least two apertures on opposite walls of the catheter tip member.

According to some embodiments, the one or more apertures include a plurality of apertures arranged in two longitudinal, or substantially longitudinal, rows on opposite walls of the catheter tip member.

According to some embodiments, the arms of the cleaning unit extend into the apertures such as to suspend the cleaning unit within the catheter tip member.

According to some embodiments, the motion of the cleaning unit includes reciprocal motion thereof along the catheter tip member, and/or tilting of the cleaning unit.

According to some embodiments, the catheter system is a ventricular catheter system for draining fluids. The fluids may include cerebrospinal fluid. The body cavity may include a brain ventricle.

According to some embodiments, the catheter system further includes a motion generator functionally associated with the controller and configured to induce the motion of the cleaning unit.

According to some embodiments, the motion generator is an electromagnet.

According to some embodiments, the cleaning unit includes or is mechanically coupled to a magnet of the electromagnet.

According to some embodiments, the motion generator is a piezoelectric motor mechanically associated with the cleaning unit.

According to some embodiments, the sensor is a magnetic sensor configured to detect changes in a magnetic field induced by the magnet, and thereby to detect the motion of the cleaning unit.

According to some embodiments, the sensor is a Hall effect sensor.

According to some embodiments, the sensor is an optical sensor.

According to some embodiments, the sensor is a proximity sensor.

According to some embodiments, the catheter system further includes a port positioned on the catheter and an implantable flexible extension connected on a first end thereof to the port and on a second end thereof to the controller, such that the connection of the flexible extension to the port forms a Y-junction.

According to some embodiments, wherein the catheter system includes a power source (e.g. a battery) as described above or a power receiver as described above, the catheter system further includes electrical wires, and/or a flex PCB with conductive tracks embedded thereon, extending along the flexible extension and a distal section of the catheter and configured to provide power to the motion generator (thus inducing motion of the cleaning unit) and/or to the sensor.

According to some embodiments, wherein the catheter system includes a piezoelectric motor as described above, the piezoelectric motor is housed in the Y-junction, or in a compartment proximally located relative thereto, or in an implantable casing housing the controller. The catheter system further includes mechanical infrastructure, extending along at least a distal section of the catheter and configured to mechanically couple the piezoelectric motor and the cleaning unit.

According to an aspect of some embodiments, there is provided a kit including a catheter system as described above, and a headset as described above.

According to an aspect of some embodiments, there is provided a method for self-monitoring the operation of a self-cleaning catheter system implanted in a body cavity of a subject. The method includes:
Providing an implanted self-cleaning catheter system as described above;
Activating the cleaning unit, thereby initiating a cleaning session.
Using the sensor to obtain one or more signals indicative of motion and/or position of the cleaning unit.
Determining whether the cleaning unit is malfunctioning, based, at least in part, on the obtained one or more signals.

According to some embodiments, the step of determining whether the cleaning unit is malfunctioning includes processing the obtained one or more signals to compute an amplitude of oscillations of the cleaning unit and/or a mean (average) position of the cleaning unit.

According to some embodiments, the step of determining whether the cleaning unit is malfunctioning includes:
Performing an initial evaluation of a malfunction in the cleaning unit, based, at least in part, on the obtained one or more signals, and if the initial evaluation indicates a malfunction:
Initiating a corrective action configured to rectify the malfunction.
Obtaining updated one or more signals indicative of motion and/or position of the cleaning unit.
Determining whether the cleaning unit is malfunctioning, based, at least in part, on the updated one or more signals.

According to some embodiments, the corrective action includes one or more of increasing a power supplied to the cleaning unit, changing a duty cycle of the cleaning unit, changing an activation waveform of the cleaning unit, changing a sampling rate of the sensor, and changing a sensitivity of the sensor.

According to some embodiments, the method further includes triggering an alert when the cleaning unit is determined to be malfunctioning.

According to some embodiments, the alert further signals that medical attention is required.

According to some embodiments, the steps of the method are repeated on a periodic basis as long as the cleaning unit is not determined be malfunctioning.

According to some embodiments, the implanted catheter system is provided as part of a kit as the kits described above.

Aspects of the disclosure, according to some embodiments, relate to a medical implant system which may be configured to provide feedback indicating that a cleaning element is actually moving within an implanted shunt. If no movement and/or improper movement is detected, an alert may be triggered.

Thus, according to an aspect of some embodiments, there is provided a self-cleaning medical device, including:
A tubular conduit configured for implantation within an anatomical body for at least one of fluid delivery, fluid drainage, and fluid passage.
An implantable moveable cleaning element associated with the tubular conduit.
An implantable sensor, associated with the cleaning element, the sensor configured to detect movement of the moveable cleaning element and to output movement signals.
An implantable transmitter configured to receive the movement signals and to transmit at least one movement indication to a receiver.

According to some embodiments, the receiver is configured for location external to the anatomical body.

According to some embodiments, the movement indication includes an indication that the cleaning element is not moving.

According to some embodiments, the movement indication includes an indicator that the cleaning element is not moving according to programmed movement commands.

According to some embodiments, the cleaning device further includes an external unit including an antenna configured to receive the at least one movement indication. According to some embodiments, the external unit includes a headset configured to be worn on a patient's head. According to some embodiments, the external unit is configured to generate an alert based on the received at least one movement indication. According to some embodiments, the alert is to seek medical attention. According to some embodiments, the transmitter is configured to transmit electromagnetic signals to the external unit.

According to some embodiments, detection of improper movement of the cleaning element may be indicative of at least partial occlusion of the shunt.

According to some embodiments, the transmitter includes an implanted antenna.

According to some embodiments, the implantable sensor is an optical sensor.

According to some embodiments, the implantable sensor is a proximity sensor.

According to some embodiments, the implantable sensor is a magnetic sensor.

According to some embodiments, the implantable sensor is an electromechanical sensor.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging”, “concluding”, “establishing”, or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A schematically depicts a prior art cerebral shunt for draining cerebrospinal fluid from a ventricle in a brain of subject;

FIG. 1B schematically depicts a prior art ventricular catheter assembly of the cerebral shunt of FIG. 1A;

FIG. 2 is a block diagram of a catheter kit including a self-cleaning, implantable catheter system, which includes a cleaning unit, and an external activation unit functionally associated with the catheter system and configured to generate an alert when the cleaning unit is malfunctioning, according to some embodiments;

FIG. 3 is a block diagram of a catheter kit including a self-cleaning, implantable catheter system, which includes a cleaning unit, and an external system communicatively associated with the catheter system and configured to generate an alert when the cleaning unit is malfunctioning, according to some embodiments;

FIGS. 4A-4C are each a flowchart of a respective protocol for monitoring performance of a cleaning unit of a self-cleaning catheter system, according to some embodiments;

FIG. 5 is a schematic, perspective view of an implantable catheter system, which is a specific embodiment of the catheter system of FIG. 2, the catheter system includes a catheter, a casing, and a flexible extension, according to some embodiments;

FIGS. 6A and 6B are schematic, perspective views of a catheter tip member and a tube distal section of a catheter similar to the catheter of FIG. 5, wherein electrical wires, extending from the catheter tip member to the casing, have been replaced by a flexible PCB strip, according to some embodiments;

FIG. 7 is a schematic, perspective view of a cleaning unit and a vibration generator of the catheter of FIG. 5, according to some embodiments;

FIG. 8 presents an output signal of a sensor monitoring the oscillatory motion of a cleaning unit within a catheter during a self-cleaning session of the catheter system of FIG. 5, according to some embodiments;

FIG. 9 is a schematic, perspective view of a catheter assembly for draining fluids, including the catheter system of FIG. 5, according to some embodiments; and

FIG. 10 schematically depicts a subject implanted with the catheter assembly of FIG. 9 and wearing a headset configured for powering the catheter system and initiating a cleaning session, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

In the description and claims of the application, the words “include” and “have”, and forms thereof, are not limited to members in a list with which the words may be associated.

In figures depicting block diagrams/flowcharts, optional elements/steps may be written within a box delineated by a dashed line.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

As used herein, according to some embodiments, a “proximal” end/section/portion/tip of an element/component/device may refer to a part of the element/component/device that is closer to a surgeon or a medical practitioner (e.g. during implantation of the device) as compared to at least one other part of the element/component/device. Similarly, according to some embodiments, a “distal” end/section/portion/tip of an element/component/device may refer to a part of the element/component/device that is further from a surgeon or a medical practitioner (e.g. during implantation of the device) as compared to at least one other part of the element/component/device. According to some embodiments, a “distal” end/section/portion/tip of an element/component/device may refer to a part of the element/component/device that is closer to a diagnosis or treatment site in the body of a patient as compared to at least one other part of the element/component/device.

As used herein, according to some embodiments, the term “implantable” with reference to an object (e.g. medical device or component/element), may refer to (i) an object which is configured to be fully implanted (e.g. a pacemaker) in the sense that when implanted no part of the object is outside the body or exposed on the skin, as well as to (ii) an object which is configured to be partially implanted (e.g. a feeding tube) in the sense that when implanted a part of the object is outside the body or exposed on the skin. According to some embodiments, an element may be said to be “implantable” when housed, or included, in another element which is implantable in the sense defined above. For example, an implantable sensor or an implantable controller may be independently implantable or implantable in the sense of being included or part of an implantable catheter.

As used herein, according to some embodiments, the term “fluid passage” is used in a broad sense to cover also one or more of fluid drainage and fluid delivery (supply).

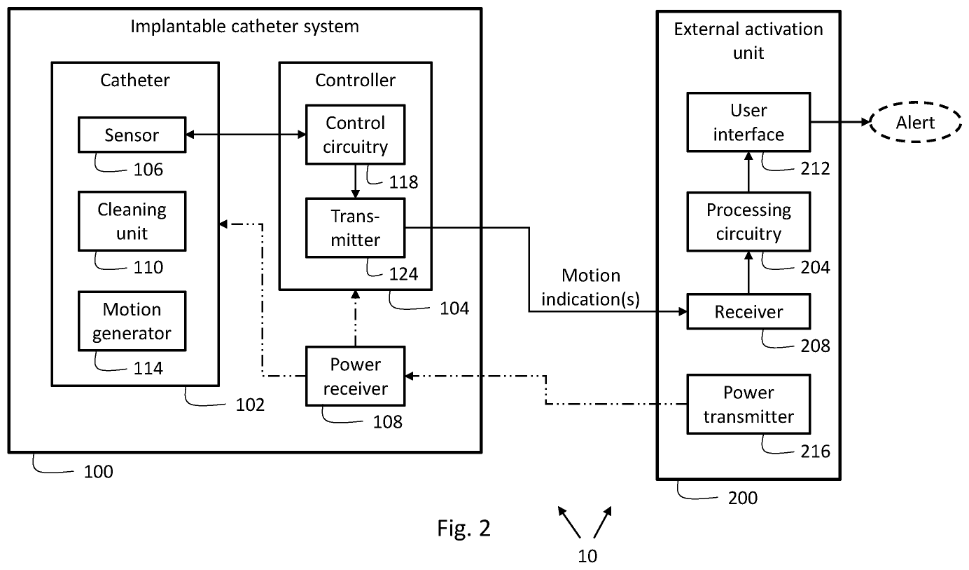

FIG. 2 is a block diagram of a catheter kit 10 including a self-cleaning, implantable catheter system 100 configured for fluid passage, and an external activation unit 200 functionally associated therewith, according to some embodiments. Catheter system 100 includes an implantable catheter 102 (or more generally, an implantable shunt and/or delivery port), an implantable controller 104, an implantable sensor 106, and an implantable power receiver 108.

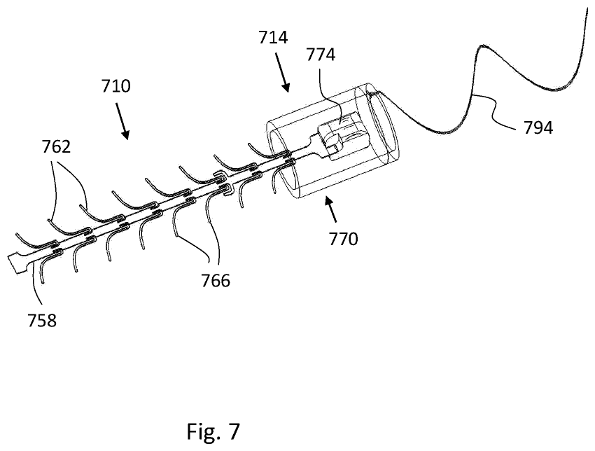

Catheter 102 is configured to be implanted in a body cavity. According to some embodiments, catheter 102 is configured to drain fluids (body fluids) from the body cavity, and/or to deliver fluids (e.g. medication) to the body cavity. Catheter 102 includes a cleaning unit 110 housed therein. Cleaning unit 110 is configured for motion (e.g. reciprocal and/or rotational motion, vibration) within catheter 102, such as to clean at least a section of catheter 102. More specifically, cleaning unit 110 is configured to mechanically prevent, or at least mitigate, blockage(s) in catheter 102, such as to maintain fluid flow through catheter 102 (or the possibility for fluid flow therethrough), as elaborated on below. According to some embodiments, and as depicted in FIG. 2, catheter 102 further includes a motion generator 114 configured to generate cleaning unit 110 motion, as explained below. According to some embodiments, motion generator 114 is mechanically associated with cleaning unit 110. According to some embodiments, motion generator 114, or a part thereof, forms part of cleaning unit 110, or is attached thereto. For example, in embodiments wherein motion generator 114 is an electromagnet, the magnet of the electromagnet may form a part of, or be attached to, cleaning unit 110, as depicted, for example, in FIG. 7 and explained in the description thereof. According to some embodiments, and as elaborated on below, motion generator 114 is a piezoelectric motor.

Sensor 106 is configured to detect cleaning unit 110 motion and to communicate to controller 104 signal(s) (i.e. one or more signals) indicative of cleaning unit 110 motion. According to some embodiments, sensor 106 is further configured to detect cleaning unit 110 position or mean position. According to some embodiments, sensor 106 is housed within catheter 102. According to some embodiments, sensor 106 is embedded in/on walls of catheter 102. According to some embodiments, sensor 106 is an optical sensor, such as a photodiode wherein the motion of cleaning unit 110 disrupts a light beam: As a non-limiting example, sensor 106 may include a light emitter and a light detector positioned on opposite sides of cleaning unit 110, such that as cleaning unit 110 moves/oscillates in the space between the light emitter and the light detector, cleaning unit 110 intermittently blocks a light beam generated by the light emitter, such that the light beam is not detected by the light detector. Alternatively, a light emitter and a light detector may be positioned on the same side of cleaning unit 110, such that as cleaning unit 110 moves/oscillates, a light beam emitted by the light emitter is intermittently reflected by cleaning unit 110, such as to be (intermittently) detected by the detector. According to some embodiments, sensor 106 is a proximity sensor. According to some embodiments, sensor 106 is a mechanical sensor. According to some embodiments, sensor 106 is an acoustic sensor. According to some embodiments, such as embodiments wherein motion generator 114 is an electromagnet, sensor 106 may be a magnetic sensor (e.g. a Hall effect sensor) configured to monitor changes in the magnetic field induced by the electromagnet, as elaborated on below.

Controller 104 is communicatively associated with both cleaning unit 110 and sensor 106. Controller 104 includes a control circuitry 118 (electronic components, processing circuitry) and a transmitter 124 (e.g. a Bluetooth or inductive antenna) communicatively associated with control circuitry 118 (e.g. by wire). Control circuitry 118 is configured to command cleaning unit 110 and sensor 106, e.g. to activate/deactivate cleaning unit 110 and/or sensor 106. According to some embodiments, control circuitry 118 and cleaning unit 110 are configured to allow controllably modifying parameters characterizing the operation of cleaning unit 110, such as the power supplied to cleaning unit 110, the duty cycle of cleaning unit 110, the activation waveform of cleaning unit 110 (e.g., the amplitude of oscillations of cleaning unit 110), etc., as expounded on below According to some embodiments, control circuitry 118 and sensor 106 are configured to allow controllably modifying parameters characterizing the operation of sensor 106, such as the sampling rate and/or the sensitivity of sensor 106. According to some embodiments, control circuitry 118 may be configured to receive the signal(s) output by sensor 106 and to convert the signal(s) into motion indication(s) (at least one motion indication), which is sent to transmitter 124. According to some embodiments, transmitter 124 may be configured to transmit the motion indication(s) to external activation unit 200, as elaborated on below.

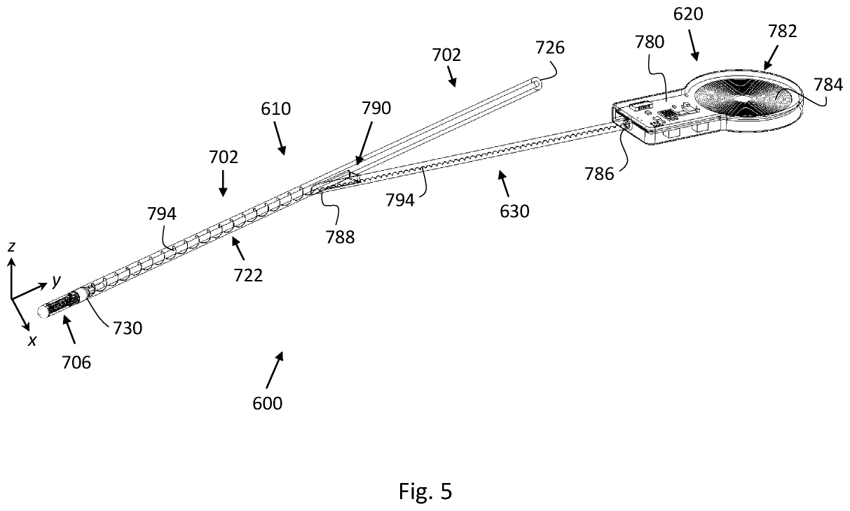

Power receiver 108 is configured to receive energy by wireless power transfer (WPT) and to power cleaning unit 110. According to some embodiments, power receiver 108 also powers controller 104, and, optionally, sensor 106. According to some embodiments, controller 104 and power receiver 108 are both housed in a common casing (such as the casing depicted in FIG. 5) that is implantable.

External activation unit 200 includes a processing circuitry 204, a receiver 208 (e.g. a Bluetooth or RF antenna), a user interface 212, and a power transmitter 216. Receiver 208 is communicatively associated (e.g. by wire) with processing circuitry 204 and user interface 212 is functionally associated with processing circuitry 204, as elaborated on below. External activation unit 200 is communicatively associated with catheter system 100. More specifically, receiver 208 is configured to receive the motion indication(s) from transmitter 124.

According to some embodiments, catheter system 100 and external activation unit 200 are configured for two-way communication there between. For example, according to some embodiments, each of transmitter 124 and receiver 208 may have both transmitting and receiving capabilities (e.g. each of transmitter 124 and receiver 208 may be a transceiver or a transmitter-receiver).

Processing circuitry 204 is configured to receive the motion indication(s) from receiver 208, and to analyze the motion indication(s) to determine whether cleaning unit 110 is functioning properly (e.g. according to programmed motion commands) or malfunctioning. According to some embodiments, processing circuitry 204 is configured, if the motion indication(s) is indicative of malfunctioning of cleaning unit 110, to command user interface 212 to output an alert. The alert signals to the subject that cleaning unit 110 is malfunctioning and may further advise the subject to seek medical attention. According to some embodiments, the alert may be aural (i.e. audible, when user interface 212 includes a speaker), visual (for example, when user interface 212 includes a display or when user interface 212 includes an indicator light(s), e.g. an LED configured to generate an alert in the form of a red light and/or a flickering/flashing light, etc.), or a combination thereof.

According to some embodiments, not depicted in FIG. 2, controller 104 does not include transmitter 124: Instead, power receiver 108 includes transmitter 124. For example, in embodiments, wherein each of power receiver 108 and power transmitter 216 includes a coil of conducting wire and are configured for WPT by inductive coupling there between, the coil in power receiver 108 may further be used for transmission of the motion indication(s) (received from control circuitry 118). According to some such embodiments, external activation unit 200 does not include receiver 208 with power transmitter 216 including receiver 208 instead.

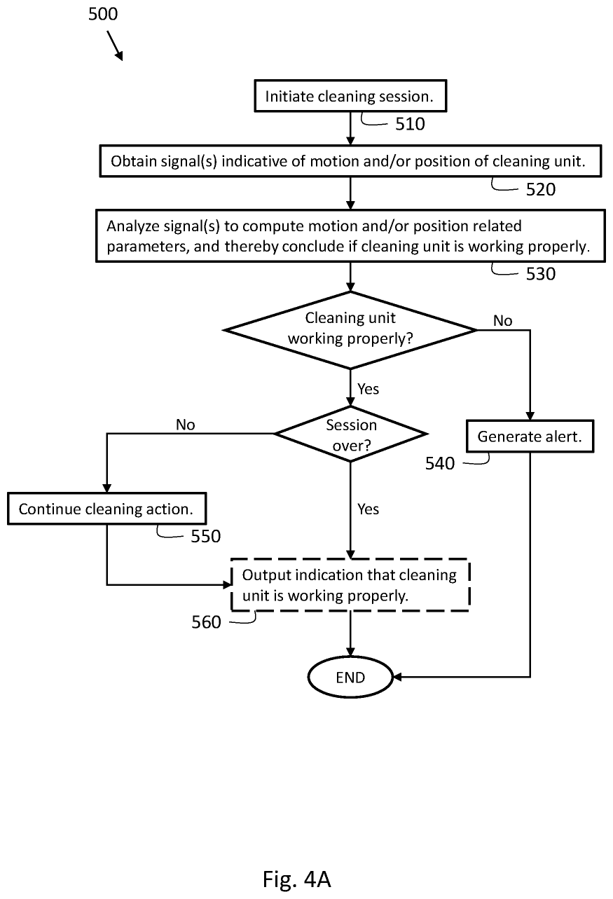

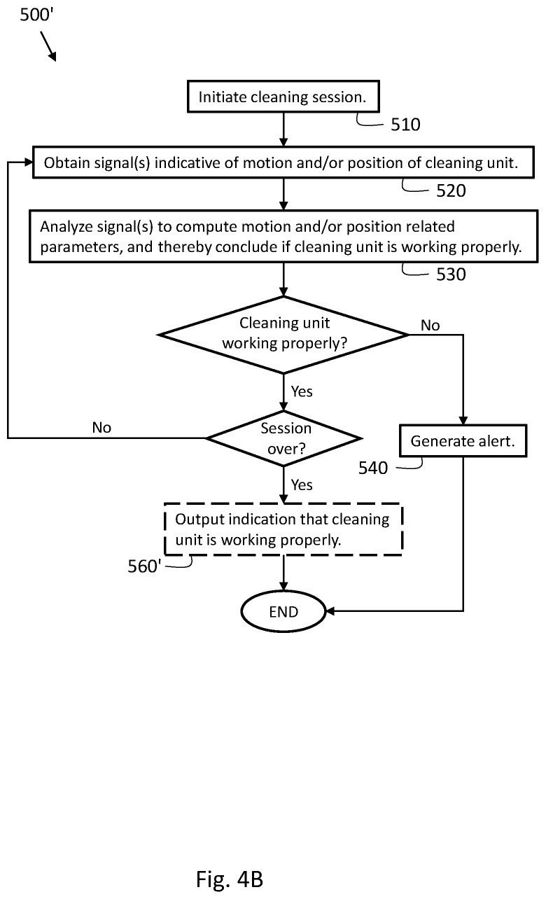

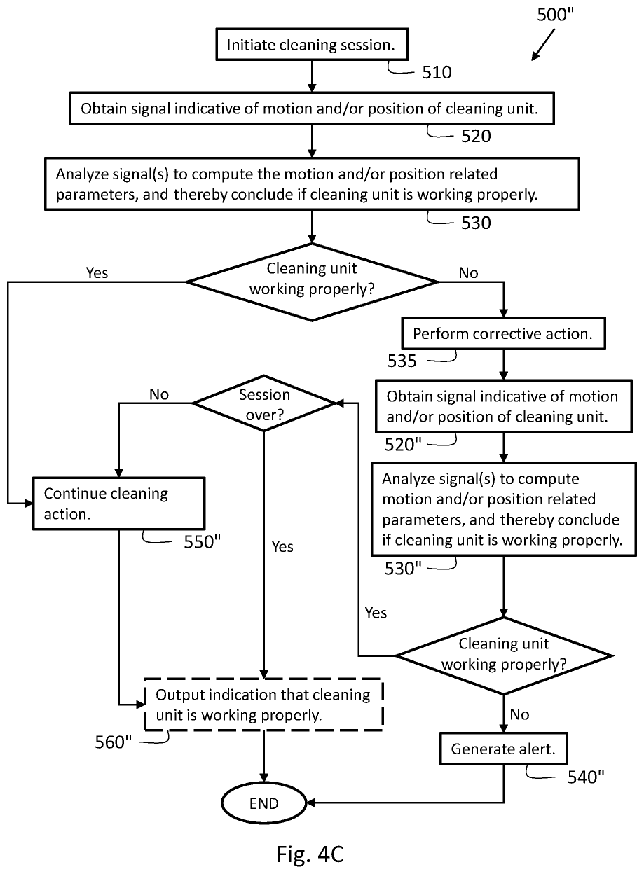

According to some embodiments (and in accordance with the monitoring protocol presented in FIG. 4C), processing circuitry 204 is configured, if the motion indication(s) is indicative of malfunctioning of cleaning unit 110, to command cleaning unit 110 to implement corrective action(s), and—only if the corrective action(s) is later determined by processing circuitry 204 to have failed to rectify the malfunctioning of cleaning unit 110—does processing circuitry 204 command user interface 212 to output an alert (essentially as described above). According to some embodiments, the corrective action(s) includes increasing power supplied to cleaning unit 110, and/or changing a duty cycle of cleaning unit 110, and/or changing the activation waveform of cleaning unit 110, and/or any combination thereof.

According to some embodiments, wherein the WPT is based on inductive coupling, each of power receiver 108 and power transmitter 216 may be a coil of conducting wire. According to some embodiments, wherein the WPT is based on capacitive coupling, each of power receiver 108 and power transmitter 216 may be a metal electrode.

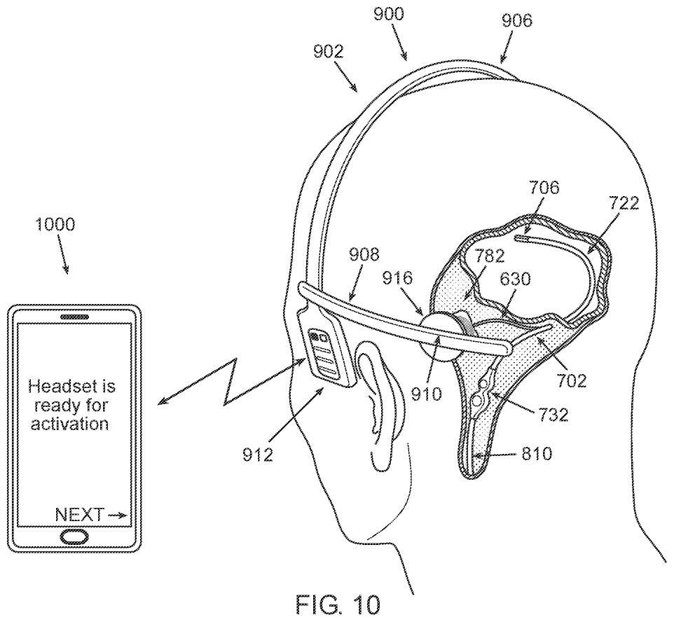

According to some embodiments, external activation unit 200 is wearable. According to some embodiments, wherein catheter system 100 is a ventricular catheter system for draining CSF fluid from a brain ventricle, external activation unit 200 is a headset configured to be worn by the subject, essentially as depicted in FIG. 10, and as elaborated on below.

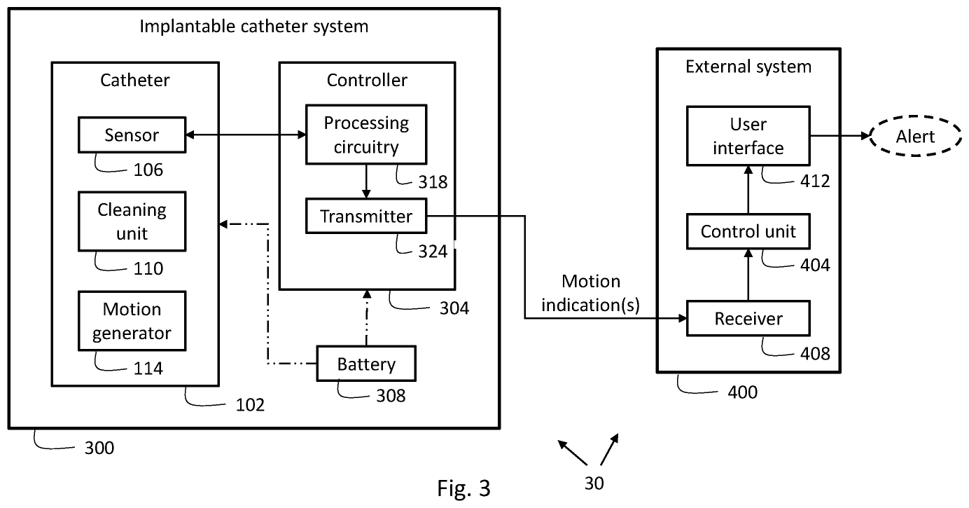

The solid lines extending between components in FIGS. 2 and 3 serve to indicate e.g. information flow and/or instructions, while the dashed-dotted lines serve to indicate e.g. transfer of power/flow of electric current from one component to another.

According to some embodiments, external activation unit 200 is functionally associated with an external device (such as the external device depicted in FIG. 10), such as a smartphone or laptop of the subject. According to some such embodiments, the alert may be generated by the external device. According to some such embodiments, the motion indication(s), received by external activation unit 200 from catheter system 100, is relayed to the external device, which performs the processing thereof to determine if cleaning unit 110 is malfunctioning.

According to some embodiments, the processing of sensor 106 signal(s), in order to determine whether cleaning unit 110 is malfunctioning, is performed in catheter system 100 by control circuitry 118 (rather by processing circuitry 204 in external activation unit 200 from the motion indication(s)). In such embodiments, control circuitry 118 includes processing circuitry configured to that end. According to some embodiments, the processing of the data is distributed, with some of the processing being performed by control circuitry 118 and some of the processing being performed by processing circuitry 204.

According to some embodiments, catheter system 100 includes at least one additional (implantable) sensor (not shown), such as a temperature sensor, a pressure sensor, a flow meter, etc. External activation unit 200 may additionally be configured to trigger an alert when the readings of the additional sensor indicate crossing of a predetermined threshold(s) and/or (sudden) change in a measured value, e.g. when the measured temperature/pressure exceeds a temperature/pressure threshold and/or increases rapidly or when the measured fluid flow rate drops below a flow rate threshold and/or changes rapidly. This alert may be different than the alert triggered due to sensor 106 readings, e.g. each of the alerts may be associated with a different sound/light pattern, etc. Similarly, when catheter system 100 includes more than one additional sensor, the alerts associated with the respective sensors may be different from one another.

It is noted that the readings of the additional sensor may also provide indication of malfunctioning of cleaning unit 110. For example, blockage/partial blockage resultant from malfunctioning of cleaning unit 110 may lead to an increase in pressure or a decrease in flow rate. Thus, according to some embodiments, processing circuitry 204 and/or control circuitry 118 may be configured to take into account the readings of the additional sensor(s) in determining whether cleaning unit 110 is malfunctioning.

FIG. 3 is a block diagram of a catheter kit 30 including a self-cleaning, implantable catheter system 300 configured for fluid passage, and an external system 400 functionally associated therewith, according to some embodiments. Catheter system 300 includes implantable catheter 102 (which includes sensor 106, cleaning unit 110, and motion generator 114), an implantable controller 304, and an implantable battery 308. Non-limiting examples of suitable batteries include implantable batteries similar to those used in pacemakers, as well as implantable batteries which are rechargeable by WPT. Catheter system 300 is similar to catheter system 100, but differs therefrom at least in being powered by battery 308 instead of by WPT (though, according to some embodiments, wherein the battery is implantable, the battery may be rechargeable by WPT).

According to some embodiments, catheter system 300 and external system 400 further differ from catheter system 100 and external activation unit 200 in that the analysis of whether the cleaning unit (i.e. cleaning unit 110) is malfunctioning is performed by catheter system 300 (i.e. by controller 304), unlike catheter system 100 and external activation unit 200, wherein the analysis is performed by external activation unit 200 (i.e. by processing circuitry 204). According to some embodiments, controller 304 and battery 308 are both housed in a common casing (not shown) that is implantable.

Controller 304 is communicatively associated with both cleaning unit 110 and sensor 106. Controller 304 includes processing circuitry 318 (processor and memory components), configured to command cleaning unit 110 and sensor 106, e.g. to activate/deactivate cleaning unit 110 and/or sensor 106. According to some embodiments, processing circuitry 318 and cleaning unit 110 are configured to allow controllably modifying parameters characterizing the operation of cleaning unit 110. According to some embodiments, processing circuitry 318 and sensor 106 are configured to allow controllably modifying parameters characterizing the operation of sensor 106. Processing circuitry 318 is configured to receive the signal(s) (indicative of cleaning unit 110 motion) output by sensor 106 and to analyze the signal(s) to determine whether cleaning unit 110 is functioning properly (e.g. according to programmed motion commands) or malfunctioning. Processing circuitry 318 is further configured to provide motion indication(s) specifying the operational state of cleaning unit 110 (e.g. whether cleaning unit 110 is malfunctioning), at least when the signal(s) (from sensor 106) is indicative of malfunctioning of the cleaning unit 110, as elaborated on below.

Controller 304 further includes a transmitter 324 (e.g. an antenna) communicatively associated with processing circuitry 318 (e.g. by wire). Transmitter 324 is configured to transmit the motion indication(s) to external system 400, as elaborated on below.

External system 400 includes a control unit 404 (including e.g. electronic circuitry, processing circuitry), a receiver 408 (e.g. an antenna), and a user interface 412. Receiver 408 is communicatively associated (e.g. by wire) with control unit 404, and user interface 412 is functionally associated with control unit 404. External system 400 is communicatively associated with catheter system 300. More specifically, receiver 408 is configured to receive the motion indication(s) from transmitter 324.

Receiver 408 is configured to relay the motion indication(s) (received from transmitter 324) to control unit 404. Control unit 404 is configured such that, if the motion indication(s) specifies malfunctioning of cleaning unit 110, to instruct user interface 412 to generate an alert. The alert signals to the subject that cleaning unit 110 is malfunctioning and may further advise the subject to seek medical attention. According to some embodiments, the alert may be aural (when user interface 412 includes a speaker), visual (when user interface 412 includes a display or an indicator light(s)), or a combination thereof.

According to some embodiments, external system 400 may be a smartphone, a smartwatch, a tablet, or a laptop having stored in the memory thereof (i.e. in control unit 404) custom software (i.e. an app) to instruct user interface 412 to generate the alert upon receiving a motion indication(s) specifying malfunctioning of cleaning unit 110. Each possibility is a separate embodiment.

According to some embodiments, the motion indication(s) is provided only when processing circuitry 318 assesses (based on the signal(s) provided by sensor 106) that cleaning unit 110 is malfunctioning. In particular, in such embodiments, transmitter 324 relays the motion indication(s) only when processing circuitry 318 has diagnosed cleaning unit 110 to be malfunctioning. According to some alternative embodiments, the motion indication(s) is provided irrespectively of the assessment of processing circuitry 318. In such embodiments, control unit 404 may further be configured to command user interface 412 to inform the subject that catheter system 100 is in order when processing circuitry 304 has concluded that cleaning unit 110 is working properly.

According to some embodiments, the processing of obtained data in order to determine whether cleaning unit 110 is malfunctioning, is performed in external system 400 by control unit 404 (rather than by processing circuitry 318 in catheter system 300). In such embodiments, control unit 404 includes processing circuitry configured to that end. According to some embodiments, the processing of the data is distributed, with some of the processing being performed by control unit 404 and some of the processing being performed by processing circuitry 318.

According to some embodiments, catheter system 300 includes at least one additional (implantable) sensor (not shown), such as a temperature sensor, a pressure sensor, a flow meter, etc., essentially as described above with respect to catheter system 100.

According to some embodiments, catheter systems 100 and 300 are ventricular catheter systems for draining fluids from ventricles, in particular, cerebrospinal fluid (CSF) from brain ventricles.

According to some embodiments of the disclosed catheter systems (e.g. catheter systems 100 and 300), the catheter systems are further configured for monitoring one or more physical parameters indicative of a condition of the subject (e.g. intracranial pressure when the catheter system is implanted in the brain) and/or proper functionality of the catheter system (e.g. fluid flow rate through the catheter). The monitoring may be performed essentially continuously (when the catheter system includes a power source) or each time a cleaning session is initiated (e.g. at least once a day). Exceeding predetermined thresholds and/or sharp changes in the measured values of the physical parameters may indicate that medical intervention is required. Trend analysis of the measured values may advantageously allow one to predict in advance the development of a physical condition (which may require medical attention). According to some embodiments, the catheter systems are further configured for self-activation (i.e. to initiate a cleaning session) on receipt of a signal indicative of occlusion in the catheter system (so that the catheter systems are configured to operate in a closed-loop manner). According to some such embodiments, the catheter system further includes an additional sensor(s), which is implantable (e.g. housed in the catheter) and configured to monitor the physical parameters. According to some embodiments, the additional sensor(s) includes a pressure sensor configured to measure the pressure within the catheter and/or the body cavity wherein the catheter is implanted. According to some embodiments, the at least one sensor(s) includes a flow meter configured to measure the fluid-flow rate (or, more generally, fluid flow related parameters) in the catheter.

FIG. 4A is a flowchart of a protocol 500 for monitoring performance of self-cleaning of a catheter system, such as catheter system 100 of catheter kit 10, catheter system 300 of catheter kit 30, or the catheter system depicted in FIG. 5, according to some embodiments. In FIGS. 4A-4C, optional steps appear within boxes delineated by dashed lines. Protocol 500 may include:
A step 510, wherein a self-cleaning session of a catheter, such as catheter 102, or a section thereof, is initiated. The self-cleaning is effected by a cleaning unit, such as cleaning unit 110, whose motion is induced by a motion generator, such as motion generator 114.
A step 520, wherein a signal(s) indicative of the motion and/or the position of the cleaning unit is obtained using a sensor monitoring the motion of the cleaning unit, such as sensor 106.
A step 530, wherein the signal(s) is analyzed (e.g. by processing circuitry such as processing circuitry 204 or processing circuitry 318) to compute the motion and/or position of the cleaning unit or quantities/parameters derived therefrom or related thereto.
A step 540, contingent (dependent) on it being concluded in step 530 that the cleaning unit is malfunctioning, wherein an alert is generated. The alert cautions the subject (and/or a caregiver thereof or medical personnel) that the cleaning unit is malfunctioning and that medical intervention may be required. The alert may be generated by a user interface, such as user interface 212 of external activation unit 200 or user interface 412 of external system 400.
A step 550, contingent on it being determined in step 530 that the cleaning unit is working properly and the cleaning session not being over (i.e. when step 530 concludes prior to the prescribed duration of the cleaning session), wherein the cleaning session is continued until its prescribed conclusion.
An optional step 560, following step 550 (or step 530 if step 530 concludes after the end of the cleaning session), wherein it is announced to the subject (and/or a caregiver thereof or medical personnel) that cleaning unit is in order.

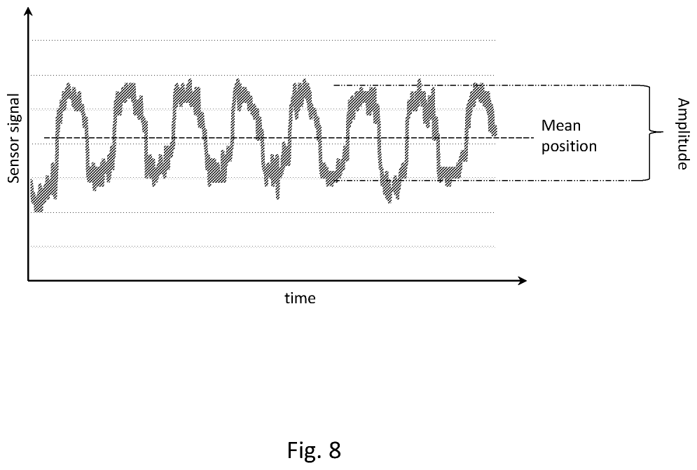

According to some embodiments, wherein the motion of the cleaning unit in the catheter is reciprocal/oscillatory, in step 530 the signal(s) (i.e. the output signal(s) of the sensor obtained in step 520) may be processed to compute the amplitude of the motion of the cleaning unit and/or the mean (average) position of the cleaning unit. An example of an output signal of a sensor of a ventricular catheter system, which is a specific embodiment of catheter system 100, is presented in FIG. 8. The amplitude is indicative of the range of motion of cleaning unit 110. Thus, a small amplitude may be indicative of limited motion due to blockage and/or a malfunction in the cleaning unit (or in other components associated thereto). The mean value is indicative of the mean position of the cleaning unit when the cleaning unit is working properly. Thus, a mean position which is displaced relative to the “normal” mean position (that is the mean position when the cleaning unit is working properly) may be indicative of unilateral blockage or partial blockage. According to some embodiments, the normal mean position of the cleaning unit when working properly may correspond to the position thereof when stationary (i.e. when switched off).

According to some embodiments, steps 520 and 530 may be repeated so long as the cleaning unit has not been diagnosed to be malfunctioning and so long as the cleaning session is not over. Some such embodiments are presented in FIG. 4B, which is a flowchart of a protocol 500′ for monitoring of performance of self-cleaning of a catheter system. Protocol 500′ is similar to protocol 500 but differs therefrom at least in that steps 520 and 530 may be repeated as described above and as illustrated in FIG. 4B. Protocol 500′ includes steps 510, 520, 530, 540, and optionally a step 560′ essentially similar to step 560 of protocol 500.

FIG. 4C is a flowchart of a protocol 500″ for monitoring performance of self-cleaning of a catheter system, such as catheter system 100 of catheter kit 10 or catheter system 300 of catheter kit 30, according to some embodiments. Protocol 500″ is similar to protocol 500 but differs therefrom at least in including a step of corrective action following a (first) diagnosis of malfunctioning of the cleaning unit. More specifically, protocol 500″ may include:
Step 510.
Step 520.
Step 530.
A step 535, contingent on it being determined in step 530 (based on the computed cleaning unit motion and/or position related parameters) that the cleaning unit is malfunctioning, wherein one or more corrective actions are performed. The corrective actions may include modifying parameters characterizing the operation of the cleaning unit, such as increasing the power supplied to the cleaning unit.
A step 520″, following step 535 and essentially similar to step 520.
A step 530″, following step 520″ and essentially similar to step 530.
A step 540″, essentially similar to step 540 of protocol 500, and contingent on it being determined in step 530″ (based on the computed motion and/or position of the cleaning unit) that the cleaning unit is still malfunctioning (i.e. that the corrective action did not rectify the malfunction), wherein an alert is generated.
A step 550″, essentially similar to step 550 of protocol 500, and contingent on it being determined in step 530 that the cleaning unit is working properly, or in step 530″ that the cleaning unit is working properly and the cleaning session not being over.
An optional step 560″, essentially similar to step 560 of protocol 500, and following step 550″ (or step 530″ if step 530″ concludes after the end of the cleaning session and the cleaning unit is determined to be working properly).

According to some embodiments, in step 535 the corrective actions include modifying parameters characterizing the motion of the cleaning unit, such as one or more of increasing the power supplied to the cleaning unit, changing the duty cycle of the cleaning unit, and modifying the activation waveform of the cleaning unit.

According to some embodiments, the sensor may measure continuously throughout the entire cleaning session. According to some embodiments, the sensor may be activated only once or twice at the beginning of the cleaning session, or at pre-determined time intervals during the cleaning session.

According to some embodiments of protocols 500, 500′, and 500″, and as depicted in the figures, the generation of the alert is followed by a cessation of the cleaning session. According to some alternative embodiments, following the generation of the alert the cleaning session continues to its full prescribed duration.

It will be understood that each of protocols 500, 500′, and 500″ may be performed by catheter kit 10, catheter kit 30, and kits similar thereto.

According to some embodiments, the determination of malfunction may be comparative/based on trend analysis: The sensor signal is compared to the last obtained sensor signal (i.e. from the previous activation), or to the last n obtained sensor signals (i.e. from the previous n activations, where n≥2), to establish whether a sudden change or a gradual deterioration in the functionality of the cleaning unit has occurred. A threshold may be set (e.g. an amplitude level or decrease range (difference in amplitude levels between activations) in the amplitude of the motion of the cleaning unit, a shift in the mean position of the cleaning unit), such that when the threshold is crossed, an alert may be generated.

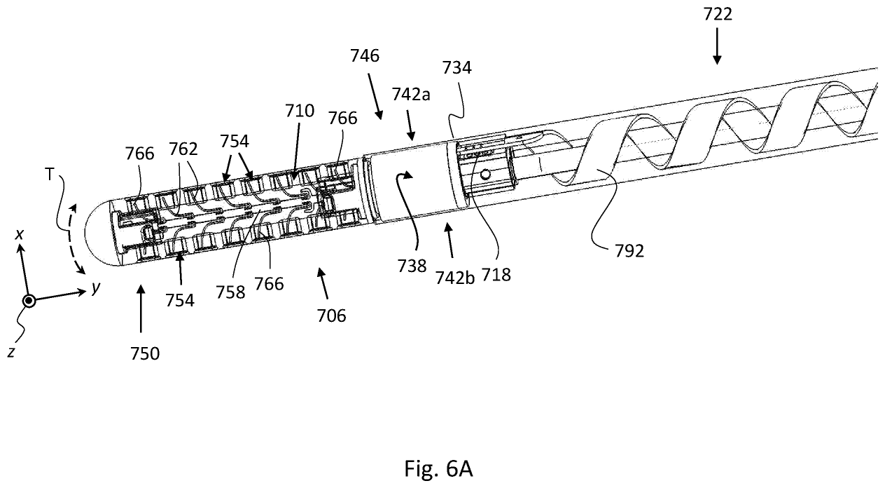

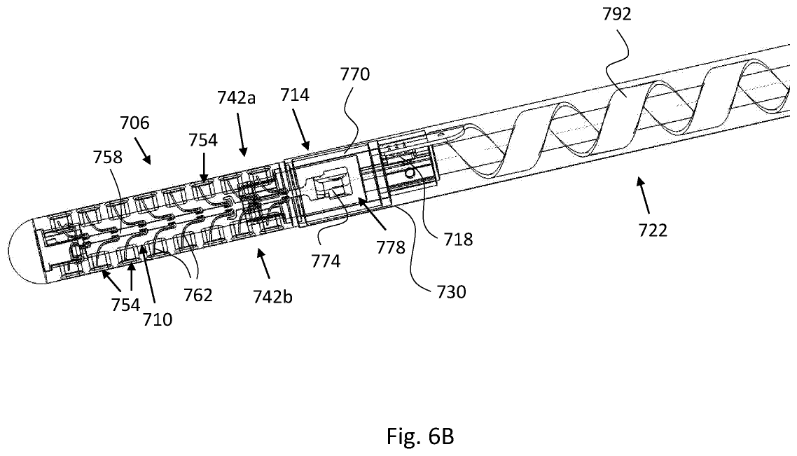

FIG. 5 is a schematic, perspective view of a catheter system 600, according to some embodiments. Catheter system 600 is a specific embodiment of catheter system 100. Catheter system 600 includes a catheter 610, a casing 620 (housing electronic circuitry and power supply components as elaborated on below), and a flexible extension 630 (e.g. a tube/cable) associating catheter 610 and casing 620, as elaborated on below. Catheter 610 is a specific embodiment of catheter 102 and includes an elongated catheter tube 702, a catheter tip member 706, a cleaning unit 710 (shown in FIGS. 6A-7), a vibration generator 714 (shown in FIGS. 6A-7), and a sensor 718 (shown in FIGS. 6A and 6B). Cleaning unit 710, vibration generator 714, and sensor 718 are specific embodiments of cleaning unit 110, motion generator 114, and sensor 106, respectively. According to some embodiments, both casing 620 and flexible extension 630 are also implantable. It is noted that, in accordance with some embodiments, flexible extension 630 and/or casing 620 may be detachable and may be connected to catheter 610 (e.g. via a port having an electrical connector; not shown) before or after the implantation of catheter 610. According to some such embodiments, catheter system 600 may be provided with flexible extensions 630 of various lengths, to accommodate different head dimensions. For example, shorter flexible extensions may be used when catheter system 600 is implanted in children and longer flexible extensions may be used when catheter system 600 is implanted in adults.

According to some embodiments, catheter system 600 is a ventricular catheter system for draining cerebrospinal fluid from a brain ventricle, and catheter 610 is configured to be implanted in a brain ventricle. According to some such embodiments, casing 620 is implantable beneath the skin but outside the skull, while flexible extension 630 is implantable (under the skull but) outside the ventricle. According to some other such embodiments, both casing 620 and flexible extension 630 are implantable beneath the skin but outside the skull.

FIGS. 6A and 6B are schematic, perspective view of a tube distal section 722 (i.e. the distal section of catheter tube 702) and catheter tip member 706, according to some embodiments. FIG. 6B differs from 6A in that in FIG. 6B some components of vibration generator 714 are outlined but otherwise depicted as transparent, as detailed below. FIG. 7 is a schematic perspective view of cleaning unit 710 and vibration generator 714.

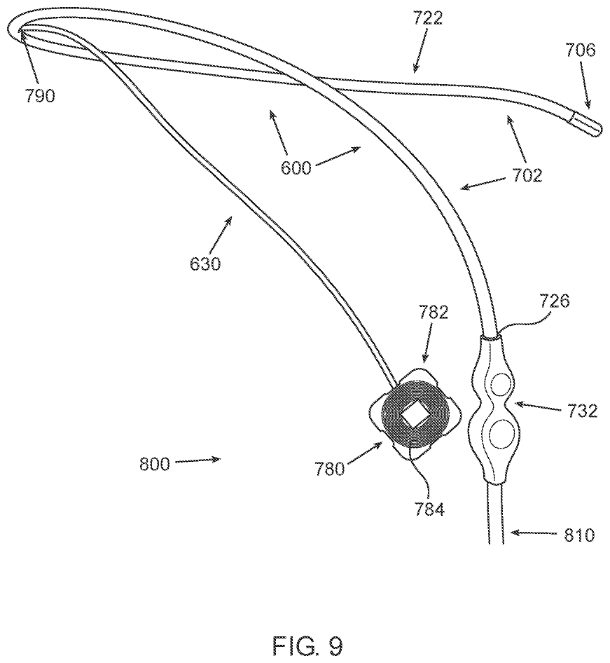

Catheter tube 702 extends from a tube proximal end 726 (shown in FIG. 5) to a tube distal end 730. Tube proximal end 726 may be configured to be connected to a valve 732 (shown in FIG. 9), which may be similar to valve 39, as elaborated on below. Tube distal end 730 is joined to catheter tip member 706, as elaborated on below.

Catheter tip member 706 is hollow (as seen in FIGS. 6A and 6B) and is open on a tip member proximal end 734 (i.e. the proximal end of catheter tip member 706), thereby being fluidly connected to catheter tube 702. According to some embodiments, catheter tip member 706 may be tubular or in the form of a short tube. Catheter tip member 706 includes a top surface 738, a bottom surface (not shown), a first side surface 742a adjacent to both top surface 738 and to the bottom surface, and a second side surface 742b opposite to first side surface 742a.

Catheter tip member 706 further includes a tip member proximal section 746 (i.e. a proximal section of catheter tip member 706; the proximal section including tip member proximal end 734) and a tip member distal section 750 (i.e. a distal section of catheter tip member 706). Tip member proximal section 746 and tip member distal section 750 are joined.

Tip member distal section 750 includes apertures 754 (not all of which are numbered) wherethrough fluids can (i) enter catheter tip member 706 from outside thereof, when the catheter is utilized for fluid drainage/passage, and (ii) exit catheter tip member 706 to the outside thereof, when the catheter is utilized for fluid delivery/passage. Tip member proximal end 734 is connected to tube distal end 730, thereby fluidly connecting apertures 754 to catheter tube 702 and allowing to (i) expel, via catheter tube 702, fluids (e.g. CSF from a brain ventricle) drained through apertures 754, or (ii) deliver, via catheter tube 702 and apertures 754, fluids (e.g. medication) to a target site/location within a subject's body. According to some embodiments, and as depicted in the figures, apertures 754 are arranged in two rows of apertures: a first row and a second row (not numbered). The two rows may extend along the length of tip member distal section 750 on opposite sides thereof, as depicted, for example, in FIGS. 6A and 6B, i.e. on first side surface 742a and second side surface 742b, respectively. According to some embodiments, apertures 754 may be round. According to some embodiments, apertures 754 may be elongated, e.g. in the form of slots.

FIG. 7 is a schematic, perspective view of cleaning unit 710 and vibration generator 714, according to some embodiments. Cleaning unit 710 (depicted also in FIGS. 6A and 6B) may be at least partially housed within tip member distal section 750. According to some embodiments, cleaning unit 714 includes a central shaft 758 and arms 762 (not all of which are numbered) extending from shaft 758, as disclosed, for example, in U.S. Pat. No. 9,393,389, titled “Self Cleaning Shunt”, to Samoocha et al., which is incorporated herein by reference in its entirety. According to some embodiments, arms 762 include two sets of arms: a first set and a second set (not numbered). According to some embodiments, shaft 758 and arms 762 span or substantially span a plane (e.g. shaft 758 and arms 762 lie or substantially lie in parallel to the xy-plane in FIG. 6A).

According to some embodiments, shaft 758 is longitudinally or substantially longitudinally disposed within catheter tip member 702. That is, shaft 758 may be disposed or substantially disposed in parallel to the y-axis (at least when cleaning unit 710 is not vibrating). According to some embodiments, arms 762 may be capable of projecting from shaft 758 such that tips 766 of arms 762 reach into apertures 754. According to some embodiments, arms in the first set are positioned such as to allow each of the arms to extend into a respective aperture from the first row of apertures (e.g. the distances between adjacent arms in the first set equal or substantially equal the distances between adjacent apertures in the first row), and arms in the second set are positioned such as to allow each of the arms to extend into a respective aperture from the second row of apertures.

According to some embodiments, shaft 758 may be configured for motion/oscillation along and/or about a longitudinal axis of catheter tip member 706. (The longitudinal axis runs parallel to the y-axis.) Arms 762 may be configured for movement (e.g. of tips 766) within apertures 754 such as to prevent tissue from entering/blocking apertures 754 and/or to remove/clear/push-out tissue which has entered/blocked one or more of apertures 754 (when catheter 610 is implanted in a ventricle, for example). According to some embodiments, shaft 758 is configured for movement (e.g. vibration) such as to induce movement of arms 762/tips 766 within apertures 754. The movement of each of arms 762/tips 766 may be such as to range over all the area of the respective aperture, so as to ensure that tissue does not penetrate into the aperture. In particular, shaft 758 may be configured for oscillatory tilting motion (as indicated by a curved double-headed arrow T in FIG. 6A), so as to effect radial movement of arms 762 within apertures 754, wherein the depth of penetration of an arm into a respective aperture alternately increases and decreases. According to some embodiments, the length(s) of arms 762 is determined according to the thickness of the walls (not numbered) of tip member distal section 750 such that tips 766 do not (e.g. cannot) protrude out of tip member proximal section 746, particularly, when cleaning unit 710 vibrates.

According to some embodiments, arms from the first set and the second set extend into apertures from the first row and the second row, respectively, thereby suspending cleaning unit 710 within catheter tip member 706 (e.g. tips 766 remain within apertures 754, in particular, when cleaning unit 710 is activated). That is, apertures 754 support cleaning unit 710 within catheter tip member 706. Further, movement of cleaning unit 710 within catheter tip member 706 is restricted, since the movement of tips 766 is restricted by the dimensions of apertures 754.

Additionally/alternatively, according to some embodiments, cleaning unit 710 may be supported/partially supported by a pin (not indicated) oriented at right angles to shaft 758 (e.g. in parallel to the z-axis) and extending through a hole (not shown) in shaft 758. The pin may function as a pivot about which shaft 758 oscillates when cleaning unit 710 is activated. The hole may be significantly larger than the pin to allow not only oscillatory tilting motion (indicated by double headed arrow T) but also reciprocal motion along the axial direction (i.e. parallel to the y-axis) to ensure that in the induced movement of arms 762, each of tips 754 covers the full length/area of the respective aperture.

Vibration generator 714 (e.g. an electromagnet or an electric or electromechanical motor) is configured to induce movement/vibration of shaft 758 (and arms 762). According to some embodiments, vibration generator 714 is mechanically coupled to cleaning unit 710. According to some embodiments, vibration generator 714 forms part of cleaning unit 710. According to some embodiments, and as depicted in FIGS. 6B and 7, some components of vibration generator 714 form part of catheter tip member 706 and other components of vibration generator 714 form part of cleaning unit 710. According to some embodiments, vibration generator 714 is an electromagnet including a coil 770 (of an electrically conducting wire) and a metallic casing 774 (shown also in FIG. 6B, wherein coil 770 is outlined but is otherwise depicted as transparent to facilitate the description). Metallic casing 774 may be or include a magnet (e.g. a neodymium magnet) and/or a magnetizable material, and may be housed in a chamber 778 inside tip member proximal portion 706. According to some embodiments, the magnet is enclosed in a corrosion-resistant metallic (e.g. titanium) casing and/or is coated with a biocompatible material. Coil 770 may be winded (wound) about a wall (not numbered, e.g. externally on the wall) of chamber 778. According to some embodiments, coil 770 is coated by an electrically-insulating material, e.g. a silicone coating or a parylene coating, or may be covered by a distal portion of catheter tube 702. Metallic casing 774 may be attached to a proximal end (not numbered) of shaft 758 such as to be at least partially disposed within coil 770.

Sensor 718 may be positioned in proximity to metallic casing 774, e.g. at a distance of no more than about 10 mm therefrom. According to some embodiments, and as depicted in FIGS. 6A and 6B, sensor 718 is located within catheter tube 702 at tube distal end 730. According to some embodiments, sensor 718 is a magnetic sensor (such as a Hall effect sensor) configured to detect changes in the magnetic field induced by the magnet enclosed in metallic casing 774 due to the generated motion of cleaning unit 710 (and metallic casing 774). According to some embodiments, sensor 718 is an optical sensor. According to some embodiments, sensor 718 is a proximity sensor.

Casing 620 includes a printed circuit board (PCB) 780, which is a specific embodiment of control circuitry 118, and a power receiver 782 (which is a specific embodiment of power receiver 108) which includes a second coil 784 of conducting wire, which, as depicted in FIG. 5, may be flat. According to some embodiments, casing 620 further includes a transmitter (e.g. an antenna; not shown) communicatively associated with PCB 780. The transmitter may be configured to send motion indication(s), received from PCB 780 and indicative of cleaning unit 710 motion, to an external activation unit, such as external activation unit 200 and specific embodiments thereof described in the description of FIG. 10. According to some embodiments, in addition to providing power, second coil 784 also serves as the transmitter.

Flexible extension 630 extends from an extension proximal end 786 (the proximal end of flexible extension 630) thereof to an extension distal end 788 (the distal end of flexible extension 630). Extension proximal end 786 is connected to casing 620, either fixedly or detachably. Extension distal end 788 may be connected to catheter tube 702, such as to form a Y-junction 790 therewith. According to some embodiments, flexible extension 630 is detachably connected to catheter tube 702.

According to some embodiments, depicted in FIGS. 6A and 6B, a flexible PCB strip 792 extends from casing 620 along flexible extension 630 and along tube distal section 722 to catheter tip member 706. According to some embodiments, depicted in FIG. 5, instead of PCB strip 792 (or in addition thereto), electrical wire/s 794 extend from casing 620 along flexible extension 630 and along tube distal section 722 to catheter tip member 706.

According to some embodiments, at least along tube distal section 722, PCB strip 792 is embedded within the walls of catheter tube 702. According to some such embodiments, at least along tube distal section 722, PCB strip 792 is winded within the walls thereof. PCB strip 792 includes conductive tracks (e.g. copper or gold tracks) electrically coupled to coil 770 and to sensor 718, on the distal end thereof (not numbered) and to second coil 784 and PCB 780 on the proximal end thereof (not numbered). PCB strip 792 is configured to supply electrical current to power vibration generator 714 and sensor 718, and to relay signals from sensor 718 to PCB 780, and, optionally, to relay commands from PCB 780 to sensor 718, as elaborated on below.

Vibration generator 714 may be activated by inducing an oscillating magnetic field through second coil 784, such as to induce an alternating current via second coil 784 and the conductive tracks running along PCB strip 792. The alternating current induces an oscillating magnetic field through coil 770 (in catheter tip member 706), which in turn induces mechanical oscillations of metallic casing 774 and cleaning element 710.

According to some embodiments, wherein instead of PCB strip 792 catheter system 600 includes electrical wires 794, electrical wires 794 are used similarly and to the same end as PCB strip 792 (e.g. to power vibration generator 714 and sensor 718).

FIG. 8 presents an exemplary signal of sensor 718 indicative of motion of cleaning unit 710 during a cleaning session. Indicated are the amplitude of the motion and the mean position of cleaning unit 710 during the oscillatory motion thereof. As mentioned above, a smaller than normal amplitude (i.e. the amplitude when there is no blockage and cleaning unit 710 is working properly) may be indicative of blockage (i.e. substantial cell growth into apertures 754 which cleaning unit 710 cannot remove), or some mechanical or electrical malfunction of cleaning unit 710 (or other components associated thereto), which is not related to blockage. Similarly, a mean position displaced relative to the normal mean position may also be indicative of blockage, or some mechanical or electrical malfunction of cleaning unit 710 (or other components associated thereto), which is not related to blockage.

According to some embodiments, not depicted in the figures, vibration generator 714 is or includes a piezoelectric motor, which is mechanically coupled to cleaning unit 710. According to some such embodiments, the piezoelectric motor is not housed in catheter tip member 706, instead being positioned more proximally. According to some such embodiments, the piezoelectric motor is housed in a compartment located at or near Y-junction 790, and is mechanically associated with cleaning unit 710 via mechanical infrastructure extending through tube distal section 722 and configured to impart the motion of piezoelectric motor to cleaning unit 710. The mechanical infrastructure may include, for example, a resilient rod/wire (the wire may be similar, or mechanically similar, to a guidewire). According to other such embodiments, the piezoelectric motor is housed in or near casing 620, and is mechanically coupled to cleaning unit 710 via mechanical infrastructure as described above (the infrastructure extending also through flexible extension 630). According to some alternative embodiments, the piezoelectric motor is housed in tube distal section 722 near tube distal end 730, or in tip member proximal section 746.

According to some embodiments, wherein catheter 610 is configured to be implanted in a brain ventricle, catheter tip member 706 is characterized by a diameter between about 2 mm and about 4 mm.

According to some embodiments, catheter tip member 706 is integrally formed. According to some embodiments, catheter tip member 706 includes, or is made of, a corrosion resistant, non-toxic, and/or non-magnetic material such as titanium.

According to some embodiments, tip member distal section 750 and tip member proximal section 746 are manufactured separately as two connectable parts (which, once assembled, are not detachable). According to some embodiments, tip member proximal section 750 and tip member distal section 746 are connected via a snap-fit mechanism (not shown). According to some embodiments, both tip member distal section 750 and tip member proximal section 746 include, or are made of, a corrosion resistant, non-toxic, and/or non-magnetic material, such as titanium. According to some embodiments, at least one of tip member distal section 750 and tip member proximal section 746 includes, or is made of, a polymeric material such as silicone. According to some embodiments, tip member proximal section 746 is made of titanium and covered with a silicone cover: over coil 770 and proximally therefrom. The silicone cover may constitute a distal portion of catheter tube 702 or constitute a dedicated silicone coating. The silicone cover may be impregnated with antibiotics, hydrophilic or hydrophobic, barium, and/or other materials as commonly used in implanted catheters.

According to some embodiments, not depicted in the figures, catheter system 600 does not include flexible extension 630. Instead, casing 620 may be housed within valve 732, or positioned in proximity thereto (e.g. on catheter tube 702 proximately to tube proximal end 726), thereby obviating the need for flexible extension 630.

According to some embodiments, a mandrel may be used to implant catheter 610, and, in particular, to guide catheter tip member 706 to an intended implantation site (e.g. within a ventricle.) According to some such embodiments, not depicted in the figures, catheter tip member 706 further includes a stopper configured to be engaged by a tip portion of the mandrel, such as to prevent the mandrel from at least one of reaching and damaging cleaning unit 710 during the implantation of catheter 610. According to some embodiments, the stopper may include a first geometrical feature (e.g. an inwardly extending flange) projecting from an inner surface of tip member proximal section 746 and the tip portion of the mandrel may include a second geometrical feature (e.g. a flange or band) radially projecting relative to a main body of the mandrel. The second geometrical feature is configured to engage the first geometrical feature, such as to allow guiding catheter tip member 706 using the mandrel.

According to such some embodiments, the stopper includes a first key pattern and the tip portion of the mandrel includes a second key pattern complementary to the first key pattern. The first and second key patterns may be configured to interlock, upon engaging of the stopper by the tip portion of the mandrel, such that a rotation of the mandrel induces an equal rotation of the catheter tip member. According to some such embodiments, the first key pattern may be configured as male and the second key pattern may be configured as female, or the first key pattern may be configured as female and the second key pattern may be configured as male.

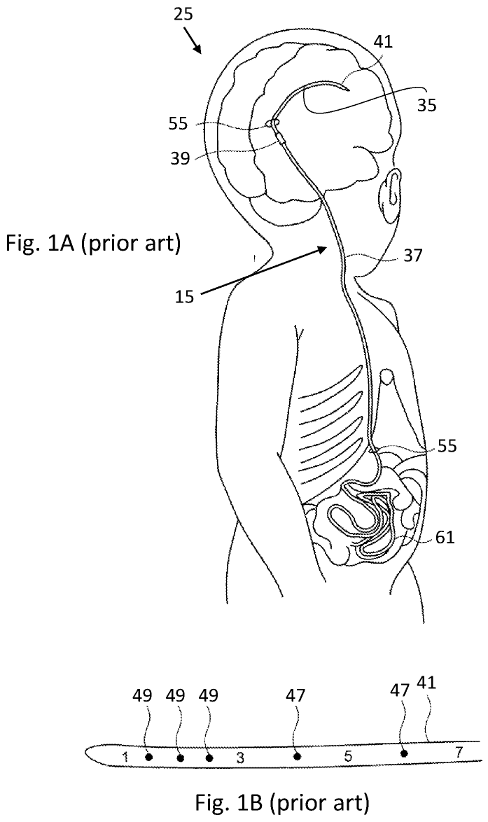

Making reference also to FIG. 9, FIG. 9 is perspective view of a catheter assembly 800 for draining body fluids, including catheter system 600 and a flexible drain tube 810, similar to drain tube 37. Drain tube 810 is fluidly coupled on an end thereof, via valve 732, to tube proximal end 726. In operation, once catheter assembly 800 is implanted in a patient (essentially as depicted in FIG. 1A), body fluids are drained via apertures 754. According to some embodiments, e.g. wherein catheter 610 is implanted in a brain ventricle and the body fluids are CSF, drained fluids may travel in the proximal direction from catheter tip member 706 into catheter tube 702, and therefrom via drain tube 810 into e.g. an abdominal cavity of the patient. More specifically, valve 732 regulates the flow of fluid from catheter tube 602 into drain tube 810. Valve 732 may be a one-way valve thereby ensuring that fluid can only flow from catheter tube 702 to drain tube 810 and not in the opposite sense (or, only in the opposite sense, in fluid delivery applications). According to some embodiments, cleaning unit 710 may be activated on a regular basis (e.g. for five minutes once a day), either manually or automatically, to ensure that apertures 754 do not become blocked by cell growth.

According to some embodiments, wherein casing 620 and flexible extension 630 are implantable, an external activation unit may be provided. The external activation unit may be configured to generate an oscillating magnetic field, so that, when operated e.g. by a patient (i.e. a subject) or a caregiver, the generated magnetic field induces an alternating current via second coil 784. FIG. 10 schematically depicts such an exemplary external activation unit 900 in the form of a headset 902 configured to be worn on the head of a subject, according to some embodiments. External activation unit 900 is a specific embodiment of external activation unit 200 of FIG. 2. More specifically, FIG. 10 schematically depicts a subject implanted with catheter assembly 800 (such that catheter tip member 706 is disposed within a brain ventricle) and wearing headset 902.

According to some exemplary embodiments, headset 902 includes an adjustable band 906, configured to secure headset 902 to the head of a subject, and an arm 908, whose function is described below. According to some embodiments, arm 908 may be pivotally connected to band 906, such as to allow maneuvering arm 908 to a configuration wherein an arm portion 910 of arm 908 is positioned adjacently to casing 620 (which is implanted in the head of the subject).

Band 906 includes processing circuitry and a receiver (not shown), and a user interface 912, which are specific embodiments of processing circuitry 204, receiver 208, and user interface 912. Arm 908 includes a power transmitter 916, which is a specific embodiment of power transmitter 216. Power transmitter 916 is positioned on arm portion 910. Band 906 may further include a rechargeable battery (not shown), or be connectable to an external power source, in order to power the processing circuitry and other electronic components of headset 902, and to supply electric current to power transmitter 916.

To initiate a cleaning session, the subject puts on headset 902 and maneuvers arm 908 such as to position arm portion 910 adjacently to casing 620, so that power transmitter 916 is adjacent to power receiver 782. The subject may then use user interface 912 to activate headset 902 (e.g. user interface 912 may include on/off buttons), so that, in particular, electrical current is supplied to power transmitter 916. Power transmitter 916 includes a coil of conducting wire (not shown), being thereby configured to transfer power to power receiver 782 via inductive coupling, so as to activate cleaning unit 710 and sensor 718, and launch a cleaning session.

According to some embodiments, power transmitter 916 may be moved along arm 908 (i.e. towards band 906 or away therefrom), thereby increasing the range of locations on the head whereon power transmitter 916 may be positioned when headset 902 is correctly worn, and thereby accounting for different head sizes (e.g. due to age) and different implant locations of casing 620.

The processing circuitry (in band 906) is configured to process motion indication(s) from PCB 780 (relayed via the receiver in band 906 and the transmitter in casing 620) to conclude if cleaning unit 710 is malfunctioning, and, optionally, to command PCB 780 to launch corrective action, as described above in the description of catheter system 100 and external activation unit 200. According to some embodiments, user interface 912 may be configured to generate, for example, an audible alert in the event that the processing circuitry has determined that cleaning unit 710 is not working properly.

According to some embodiments, headset 902 may be communicatively associated with an external device 1000, such as a smartphone (as depicted in FIG. 10), a tablet, or a laptop of the subject, which may be used to activate headset 902 and/or to generate the alert in the event of a malfunction. Additionally or alternatively, headset 902 may be configured to transmit the motion indication(s), received from catheter system 600, to the external device, which processes the motion indication(s) to determine if cleaning unit 710 is malfunctioning.

According to some alternative embodiments, the determination of whether cleaning unit 710 is malfunctioning is performed by PCB 780 (rather than in headset 902 based on the motion indication(s) received from PCB 780). According to such embodiments, PCB 780 is configured to compute the amplitude of the motion and the mean position of cleaning unit 710 from the signal(s) received from sensor 718, and, based thereon, to conclude if cleaning unit 710 is malfunctioning.

According to some embodiments, there is provided an external activation unit, which is a specific embodiment of external activation unit 200, configured as, or for use with, a commercial headset e.g. for listening to music. According to some such embodiments, the external activation unit includes a mountable arm similar to arm 908 (and including a power transmitter similar to power transmitter 916) configured to be mounted on, or removably attached to, the headset. According to some embodiments, a user interface, similar to user interface 912 and associated with the power transmitter, may also be mounted on the headset. According to some embodiments, the user interface may be included in the mountable arm. According to some embodiments, the arm includes processing circuitry and a wireless communication unit and is configured to be operated using an external system, such as a smartphone.

As used herein, according to some embodiments, the terms “control circuitry” and “processing circuitry” may be used interchangeably.

The skilled person will understand that when referring to computational functions as being “performed” by PCB 780, it is actually electronic/control/processing circuitry included in PCB 780, which performs these functions.

The skilled person will understand that when stating, for example, that “the power to the cleaning unit is increased”, according to some embodiments, what is meant is that the power supplied to the motion generator is increased (e.g. the power supplied to the coil, of the electromagnet, so as to induce motion of the magnet (of the electromagnet) which may form a part of the cleaning unit).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1-31. (canceled)

32. A self-cleaning catheter system with self-monitoring capabilities, the catheter system comprising:
a catheter configured to be implanted in a body cavity of a subject;
a cleaning unit configured for motion within the catheter such as to mechanically prevent, remove and/or mitigate blockage of at least a section of the catheter;
an implantable sensor; and
an implantable controller functionally associated with the cleaning unit and configured for activation thereof,
wherein the implantable sensor is communicatively associated with the implantable controller and is configured to detect motion of the cleaning unit, when the cleaning unit is activated, and to output one or more signals indicative of the motion of the cleaning unit; and
wherein the implantable controller is configured to receive the one or more signals from the implantable sensor and, based, at least, thereon, to provide at least one motion indication, at least when the one or more signals are indicative of malfunctioning of the cleaning unit.

33. The catheter system of claim 32, wherein the malfunctioning of the cleaning unit comprises at least one of the cleaning unit not moving, the cleaning unit not moving according to programmed motion commands, and the cleaning unit being out of position.

34. The catheter system of claim 32, wherein the catheter system is a ventricular catheter system for draining fluids, wherein the fluids comprise cerebrospinal fluid, and wherein the body cavity comprises a brain ventricle.

35. The catheter system of claim 32, wherein the sensor is housed in the catheter.

36. The catheter system of claim 32, further comprising an implantable power receiver configured for receiving wireless power transfer from an external activation unit, the implantable power receiver being further configured to power one or more of the implantable controller, the cleaning unit, and the implantable sensor.

37. The catheter system of claim 36, wherein the implantable controller comprises a transmitter configured to transmit the at least one motion indication to one or more of the external activation unit and an external system, the one or more of the external activation unit and the external system being configured to generate an alert when the at least one motion indication indicates the malfunctioning of the cleaning unit.

38. The catheter system of claim 32, wherein the implantable controller comprises a processing circuitry configured to assess whether the cleaning unit is malfunctioning based, at least in part, on the one or more signals received from the implantable sensor, and wherein the at least one motion indication specifies the assessment.

39. The catheter system of claim 38, wherein the one or more signals output by the implantable sensor comprise at least two signals comprising a first signal and a later last signal;
wherein the processing circuitry is further configured to assess whether the cleaning unit is malfunctioning upon receiving the first signal, and, if the assessment based, at least in part, on the first signal, is of malfunctioning, to initiate corrective action; and
wherein the processing circuitry is further configured to assess again whether the cleaning unit is malfunctioning upon receiving the last signal, and wherein the at least one motion indication specifies the assessment based, at least in part, on the last signal.

40. The catheter system of claim 38, wherein the processing circuitry is configured to compute, based on the received one or more signals, an amplitude of oscillations of the cleaning unit and/or a mean position of the cleaning unit, and wherein the assessing is based, at least in part, on the amplitude and/or the mean position.

41. The catheter system of claim 37, wherein one or more of the external activation unit and the external system comprises a processing circuitry configured to assess whether the cleaning unit is malfunctioning based, at least in part, on the at least one motion indication.

42. The catheter system of claim 37, wherein the external system is one or more of: a smartphone, smartwatch, tablet, laptop, PC, or cloud computer.

43. The catheter system of claim 37, wherein the external activation unit is a headset configured to be worn by the subject, the headset comprising at least one user interface configured to generate the alert and to allow the subject to activate the cleaning unit.

44. The catheter system of claim 32, wherein the implantable sensor is configured to be one of (i) automatically activated upon activation of the cleaning unit and (ii) manually activated.

45. The catheter system of claim 32, further comprising an implantable power source configured for powering one or more of the implantable controller, the cleaning unit, and the implantable sensor.

46. The catheter system of claim 32, wherein the catheter comprises a catheter tube and a catheter tip member fluidly connected to the catheter tube and housing the cleaning unit, and wherein the catheter tip member comprises one or more apertures fluidly coupling the catheter tube to the outside thereof and wherein the cleaning unit comprises an elongated shaft comprising one or more arms configured to project into the one or more apertures and to move therein, and wherein the movement of the one or more arms is induced by vibration of the cleaning unit.

47. The catheter system of claim 46, wherein the motion of the cleaning unit comprises reciprocal motion thereof along the catheter tip member, and/or tilting of the cleaning unit.

48. The catheter system of claim 32, further comprising a motion generator functionally associated with the implantable controller and configured to induce the motion of the cleaning unit.

49. The catheter system of claim 48, wherein the motion generator is an electromagnet, wherein the cleaning unit is mechanically coupled to a magnet of the electromagnet, and wherein the implantable sensor comprises a magnetic sensor configured to detect changes in a magnetic field induced by the magnet, and thereby to detect the motion of the cleaning unit.

50. The catheter system of claim 32, wherein the implantable sensor comprises at least one of an optical sensor or a proximity sensor.

51. A method for self-monitoring the operation of a self-cleaning catheter system implanted in a body cavity of a subject, the method comprising:
providing an implanted self-cleaning catheter system comprising:
a catheter configured to be implanted in a body cavity of a subject;
a cleaning unit configured for motion within the catheter such as to mechanically prevent, remove and/or mitigate blockage of at least a section of the catheter;
an implantable sensor configured to detect motion of the cleaning unit, when the cleaning unit is activated, and to output one or more signals indicative of the motion of the cleaning unit; and
an implantable controller functionally associated with the cleaning unit and configured for activation thereof, the implantable controller being further communicatively associated with the implantable sensor;
activating the cleaning unit, thereby initiating a cleaning session;
obtaining one or more signals indicative of motion and/or position of the cleaning unit, using the implantable sensor; and
determining whether the cleaning unit is malfunctioning, based, at least in part, on the obtained one or more signals.

52. The method of claim 51, wherein the step of determining whether the cleaning unit is malfunctioning comprises processing the obtained one or more signals to compute an amplitude of oscillations of the cleaning unit and/or a mean position of the cleaning unit.

53. The method of claim 51, wherein the step of determining whether the cleaning unit is malfunctioning comprises:
performing an initial evaluation of a malfunction in the cleaning unit, based, at least in part, on the obtained one or more signals;
if the initial evaluation indicates a malfunction,
initiating a corrective action configured to rectify the malfunction;
obtaining updated one or more signals indicative of motion and/or position of the cleaning unit; and
determining whether the cleaning unit is malfunctioning, based, at least in part, on the updated one or more signals.

54. The method of claim 53, wherein the corrective action comprises one or more of: increasing a power supplied to the cleaning unit, changing a duty cycle of the cleaning unit, changing an activation waveform of the cleaning unit, changing a sampling rate of the implantable sensor, and changing a sensitivity of the implantable sensor.
